(19) United States
(12) Patent Application Publication
(10) Pub. No.: US 2010/0179346 A1
(43) Pub. Date: Jul. 15, 2010

(54) METHOD FOR HYDROGENATING GLYCEROL

(76) Inventors: Norbert Klein, Mettmann (DE); Alfred Westfechtel, Hilden (DE); Martin Bähr, Langenfeld (DE)

Correspondence Address: SMITH MOORE LEATHERWOOD LLP, P.O. BOX 21927, GREENSBORO, NC, 27420, US

(21) Appl. No.: 12/602,913

(22) PCT Filed: Jun. 11, 2008
(86) PCT No.: PCT/EP2008/004666

§ 371 Date: Mar. 2, 2010

(30) Foreign Application Priority Data
Jun. 11, 2007 (DE) 10 2007 027 372.1

(51) Int. Cl. C07C 67/00; C07C 29/17; C07C 31/20; B01J 19/00; C07C 41/01; C07C 209/00; C07C 249/00

(52) U.S. Cl. 560/129; 568/861; 422/188; 568/671; 564/463; 560/336

(57) ABSTRACT

The present invention relates to a process for preparing 1,2-propanediol by hydrogenation of glycerol by means of hydrogen gas, wherein glycerol is reacted with hydrogen in at least “i” fluidically interconnected reactors R₁ to Rᵢ each having a hydrogenation catalyst to form 1,2-propanediol, wherein the process comprises sequential steps from reactors R₁ through Rₙ, wherein n as used hereinafter is an integer in the range from 2 to i. The process includes introducing hydrogen gas and a glycerol phase into the first reactor R₁ and a first 1,2-propanediol containing phase and a first hydrogen phase are formed in the reactor, and introducing sequentially the 1,2-propanediol containing phase formed in the preceding reactor and hydrogen into each of the subsequent reactors wherein the glycerol phase containing at least 60% by weight, based on the total weight of the glycerol phase of glycerol.

The present invention relates to a process for preparing 1,2-propanediol by hydrogenation of glycerol by means of hydrogen gas, to the 1,2-propanediol obtainable using this process, to a device for preparing 1,2-propanediol and to a process for preparing a compound having at least one ether group, at least one ester group, at least one amino group, at least one urethane group, or at least two thereof.

1,2-Propanediol is an industrially interesting raw material. It is used in a large number of applications, namely in the food industry, as a solvent for dyes and aroma substances, as a moisture retaining agent for tobacco, in cosmetics, as a component of brake and hydraulic fluids, antifreeze agents, lubricants in refrigerating machines, as a solvent for fats, oils, resins, waxes, dyes, etc. It also serves as a starting product for preparing other products. Numerous products which can be used as solvents, for syntheses, as softeners, thickening agents, emulsifiers, etc. can be obtained by esterification and/or etherification. In the majority of the aforementioned applications, the purity of the 1, 2-propanediol plays a part. 1, 2-Propanediol is conventionally prepared industrially by hydration of propylene oxide. In addition to the hydration of propylene oxide, the hydrogenation of glycerol is also an example of a possible process for preparing 1,2-propanediol, although this process has to date not been able to become established in industrial practice.

Processes known in the art for preparing 1,2-propanediol by hydrogenation of glycerol are described for example in DE-A-43 02 464. According to the teaching of this document, glycerol is continuously hydrogenated in the presence of a specific heterogeneous catalyst in tubular reactors or multitube fixed-bed reactors, which are operated preferably under isothermal conditions. In the examples of this document a single, two meter-long reaction tube which has an inner diameter of 25 mm is used, which is filled with copper chromite tablets. A mixture of hydrogen gas and a glycerol phase is passed through this reaction tube at a pressure in a range of from 50 to 250 bar and a temperature in a range of from 180 to 270° C.

However, the drawback of the process described in DE-A-43 02 464 consists inter alia in the fact that the glycerol is reacted to form 1,2-propanediol with comparatively low selectivity and, in addition to 1,2-propanediol, numerous by-products, such as for example n-propanediol, propanediol or methanol are formed, which subsequently have to be separated off from the target product (1,2-propanediol) via complex purification processes.

Also known in the art is the use of multitube fixed-bed reactors for hydrogenating organic compounds, such as for example fats, fatty acids or fatty acid methyl esters. However, the drawback of using multitube fixed-bed reactors of this type is that, owing to the relatively small cross section of the reaction tubes arranged in a multitube fixed-bed reactor of this type, the total catalyst volume is comparatively small. Owing to the small catalyst volume, the catalyst is therefore rapidly consumed, so that when multitube fixed-bed reactors of this type are used for hydrogenating organic compounds, the catalyst bed has to be regenerated at comparatively short intervals of time.

The object of the present invention was to overcome, or at least to reduce, the drawbacks resulting from the prior art in the preparation of 1,2-propanediol by hydrogenation of glycerol.

In particular, the present invention was based on the object of specifying a new process for preparing 1,2-propanediol by hydrogenation of glycerol, in which fewer by-products are formed than in the processes known in the art.

Furthermore, this process should have the advantage of being able to be economically operated for as long as possible, before the device used in this process for preparing the 1,2-propanediol has to be shut down for the purposes of regenerating the catalyst.

A contribution to achieving the objects mentioned at the outset is provided by a process for preparing 1,2-propanediol by hydrogenation of glycerol by means of hydrogen gas, wherein glycerol is reacted with hydrogen in at least i fluidically interconnected reactors R₁ to Rᵢ each having a hydrogenation catalyst to form 1,2-propanediol, wherein
hydrogen gas and a glycerol phase P_{glycerol} are introduced into the first reactor R₁ and a first, preferably liquid 1,2-propanediol containing-phase P₁ and a first, preferably gaseous hydrogen phase H₁ are formed in the reactor R₁,
the preferably liquid 1,2-propanediol containing phase Pₙ₋₁ formed in the preceding reactor Rₙ₋₁ and hydrogen are introduced into each of the subsequent reactors Rₙ, n to being an integer from the range of from 2 to i, and a preferably liquid 1,2-propanediol containing phase Pₙ and a preferably gaseous hydrogen phase Hₙ are formed in the reactor Rₙ,
   wherein the glycerol phase P_{glycerol} containing at least 60% by weight, particularly preferably at least 80% by weight, even more preferably at least 90% by weight and most preferably at least 97% by weight, based on to total weight of the glycerol phase P_{glycerol}, of glycerol.

In this case, it is particularly preferred that each 1,2-propanediol containing phase Pₙ is cooled
i) within each reactor Rₙ,
ii) in a region between the reactor Rₙ and Rₙ₊₁, or
iii) within each reactor Rₙ and in a region between the reactor Rₙ and the reactor Rₙ₊₁, alternative iii) being particularly preferred.

It has been found completely surprisingly, but none the less advantageously, that glycerol-rich starting compositions can be hydrogenated particularly selectively in a cascade reactor if the product mixtures issuing from the reactors are cooled before they enter the subsequent reactor and/or within the individual reactors. If the reactors used are tubular reactors, the process according to the invention can in addition be operated for a very long time before the catalyst used in the reactors for hydrogenating the glycerol has to be regenerated.

In the first step of the process according to the invention, a glycerol phase P_{glycerol} is introduced into the first reactor R₁, the glycerol phase P_{glycerol} containing at least 60% by weight, particularly preferably at least 80% by weight, even more preferably at least 90% by weight and most preferably at least 97% by weight, based on the total weight of the glycerol phase P_{glycerol}, of glycerol. In addition to the glycerol, the glycerol phase P_{glycerol} also contain further components, such as for example glycerol-based fatty acid esters which are cleaved during the hydrogenation so as to form fatty acids and glycerol, fatty alcohols also being formed in this case in addition to the 1,2-propanediol. It is however preferred according to the invention that the glycerol phase P_{glycerol} contains less than 50% by weight, particularly preferably less than 25% by weight, even more preferably less than 10% by weight, additionally preferably less than 5% by weight, based in each case on the total weight of the glycerol phase P_{glycerol}, and most preferably no glycerol-based fatty acid esters at all.

Furthermore, it is according to the invention preferred that the glycerol phase P_{glycerol} entering the reactor R₁ has a temperature in a range of from 140 to 260° C., particularly preferably in a range of from 160 to 240° C. and most preferably in a range of from 180 to 220° C.

As the hydrogenation catalyst in the reactor R₁ and preferably also in the subsequent reactors R₂ to Rᵢ full and carrier contacts containing as their main component metals, metal salts or metal oxides or the like from subgroups I and VIII can be used. Further metals can be added as dopants to improve the properties. Preferably a heterogeneous, particularly preferably a copper-containing catalyst, and additionally preferably a heterogeneous, copper and chromium-containing catalyst is used. Catalysts of this type can be produced in different ways. Particularly relevant are precipitation of the metal salts, in particular of the copper salts, impregnation, ion exchange or solid-state reactions, to name but a few examples. Catalysts which are particularly preferably used in accordance with the invention are in particular catalysts containing Cu chromite, Cu zinc oxide, Cu aluminum oxide or Cu silicon dioxide, Cu chromite-containing catalysts being most preferred.

The Cu chromite catalyst which is preferably used in this context contains 35 to 55% by weight, preferably 40 to 50% by weight of copper, 35 to 55% by weight, preferably 40 to 50% by weight of chromium, based in each case on the oxidic catalyst mass, and optionally further alkaline-earth or transition metals, in particular barium and manganese, in the form of the oxides thereof. It is in this case beneficial if the catalyst contains 1 to 7% by weight, in particular 1.5 to 3% by weight of barium, based on the oxidic catalyst mass. As an example of a suitable catalyst a catalyst shall be mentioned that contains approximately 47% by weight of CuO, 46% by weight of Cr₂O₃, 4% by weight of MnO₂ and 2% by weight of BaO. This catalyst and the process for the production thereof are described in detail in EP 254 189 A2. Reference is hereby expressly made to the disclosure contained in EP 254 189 A2, and the information provided therein should also form part of the present application. The invention is not however limited to Cu chromite catalysts. Other catalysts, such as for example Cu/ZnO catalysts or Cu/Al₂O₃ catalysts, can also be used. Catalysts which are suitable for the process according to the invention are commercially available from Südchemie AG, Germany, and Engelhard Inc., USA.

It is furthermore preferred that the catalyst has a high surface area and porosity, thus providing high activity and selectivity and a long service life which is particularly important for technical applications. Thus, it is advantageous if the catalyst used has a specific surface area in the range of from 20 to 100 m²/g, preferably 70 to 80 m²/g.

The reactors R₁ to Rᵢ which are used can be all reactors which are known to a person skilled in the art and are configured to allow hydrogenation of glycerol by means of hydrogen gas under the pressure and temperature conditions required therefore, the use of tubular reactors being most preferred. Also conceivable is the use of reactors having heat exchange plates as constructional elements. Both in the tubular reactors and in the reactors having heat exchange plates, the catalyst can be introduced in the form of a catalyst fixed-bed charge or else be attached as a coating to the inside of the tubes or heat exchange plates. It is in this connection preferable if at least one of the reactors R₁ to Rᵢ preferably all reactors R₁ to Rᵢ, has/have a catalyst charge.

Furthermore, it is according to the invention particularly preferred that the hydrogenation in at least one of the reactors R₁ to Rᵢ preferably in all reactors R₁ to Rᵢ is carried out in such a way that the phase entering the respective reactor Rₙ (the glycerol phase P_{glycerol} for the reactor R₁ or the 1,2-propanediol containing phases Pₙ₋₁ in the reactors R₂ to Rᵢ) is passed in the form of liquid fluids in trickle bed operation in parallel or countercurrent flow with hydrogen over a catalyst fixed bed. In a further embodiment using reaction tubes, it is preferred that the glycerol phase P_{glycerol} entering the reactor R₁ and the 1,2-propanediol containing phase Pₙ₋₁ entering the reactors R₂ to Rᵢ, n being an integer from the range of from 2 to i, are passed through the catalyst charge with little backmixing for a defined residence time. In one particular embodiment of the process according to the invention using reaction tubes as reactors R₁ to Rᵢ, the glycerol phase P_{glycerol} or the subsequent 1,2-propanediol containing phases Pₙ is/are passed through the catalyst charge in the reaction tube or tubes under measures at least partly preventing backmixing at an LHSV (“Liquid Hourly Space Velocity”), expressed in m³/h of glycerol per m³ of catalyst volume, in a range of from 0.1 to 20 h⁻¹, preferably in a range of from 0.1 to 5 h⁻¹, even more preferably in a range of from 0.2 to 3 h⁻¹ and additionally preferably in a range of from 0.3 to 2 h⁻¹. Examples of measures at least partly preventing backmixing include in principle all measures which are known to a person skilled in the art and seem suitable to him for this purpose, such as suitable tube cross sections or tube cross section/length ratios which are usually selected as a function of the flow conditions conventionally prevailing during operation of the reactor.

In addition to the glycerol phase P_{glycerol}, hydrogen gas is also introduced into the first reactor. In this case it is in principle possible to feed in the hydrogen exclusively from above or else exclusively from below (in the case of a perpendicular arrangement of the reactor R₁) or exclusively from the left or exclusively from the right (in the case of a horizontal arrangement of the reactor R₁). It is however also conceivable to feed in the hydrogen via a plurality of, preferably via two, three, four, five, six or more feed points from the inside directly into the catalyst fixed bed or else via a plurality of, preferably via two, three, four, five, six or more lances, from the outside directly into the catalyst fixed bed. Also conceivable is a combination of at least two of the above-described alternatives for feeding in the hydrogen gas. However, feeding of the hydrogen into the reactor from above is preferred. Furthermore, the amount of fed-in gas can be regulated via the feed points or lances by means of suitable regulating means, preferably by means of a valve. For a preselected amount of hydrogen, for example a constantly operating circulating gas pump, the hot gas/cold gas distribution can be adapted by means of valves to the respective catalyst activity and exothermy. In addition to regulation of the amount of the cold hydrogen gas via regulating means such as valves, the distribution of the fed-in amount of gas can in particular also be regulated via other or further measures, such as for example the selection of a suitable diameter of the holes inside the reactor through which the hydrogen gas enters.

Furthermore, it is according to the invention preferred that the hydrogen gas fed into the reactor R₁ has a temperature in a range of from 140 to 260° C., particularly preferably in a range of from 160 to 240° C. and most preferably in a range of from 180 to 220° C.

It is in addition in principle possible and also according to the invention particularly preferred to feed hydrogen gas together with the glycerol phase P_{glycerol} into the reactor, wherein in this case the mixture of glycerol and hydrogen gas can preferably be fed in by way of the above-described feed variants. The advantage of the common feeding of the hydrogen gas and the glycerol phase P_{glycerol} consists in particular in the fact that these two phases can together be heated to the above-mentioned temperatures in a single continuous-flow heater prior to entering the reactor R₁. However, separate feeding of the glycerol phase P_{glycerol} and the hydrogen phase is in principle also conceivable, the glycerol phase being fed in for example from above and the hydrogen gas from below, from outside via lances or from inside via a plurality of feed points directly into the catalyst bed.

The hydrogen gas used in the first reactor may in principle be fresh hydrogen, however the use of what is known as a “circulating gas” is preferred. This “circulating gas” is understood to be hydrogen which has issued from one of the subsequent reactors R₁ to Rᵢ, preferably from the last reactor Rᵢ, as a hydrogen phase Hᵢ. This hydrogen can, after cooling and subsequent separating-off from the 1,2-propanediol containing phase Pᵢ, for example after mixing with fresh hydrogen, which compensates for the reaction and loss amounts, and passing through a circulating gas pump, which compensates for the losses in pressure, again be used as hydrogen in the first reactor. Obviously, it is also conceivable to use a mixture of circulating gas and fresh hydrogen gas as the hydrogen gas in the reactor R₁.

Furthermore, in the process according to the invention, diluted or undiluted hydrogen can in principle be used in the reactor R₁. Furthermore, it is preferred that in the process according to the invention a molar ratio of hydrogen to glycerol in the range of from 2 to 500, particularly preferably 10 to 350 is set. This means that the amount of hydrogen gas throughput through the first reactor, measured in mol of H₂/hour, is 2 to 500 times higher than the amount of glycerol throughput through the first reactor, measured in mol of glycerol/hour. A most preferred range of the reaction ratio is from 30:1 to 200:1.

The pressure in at least one of the reactors R₁ to Rᵢ, preferably in all reactors R₁ to Rᵢ, is preferably in the range of from 20 to 300 bar, in particular 100 to 280 bar, whereas the temperature in at least one of the reactors R₁ to Rᵢ, preferably in all reactors R₁ to Rᵢ is preferably in a range of from 150° C. to 280° C., in particular 200 to 250° C.

Furthermore, it is according to the invention preferred that the glycerol in the first reactor R₁ is reacted preferably to 40 to 80 mol %, particularly preferably to 45 to 65 mol %, even more preferably to approximately 50 mol %.

After reacting the glycerol in the reactor R₁ under the reaction conditions described hereinbefore, there is formed, in addition to the non-reacted hydrogen gas (=hydrogen phase H₁), in the reactor R₁ a preferably liquid 1,2-propanediol containing phase P₁ which is introduced into the subsequent reactor R₂ preferably unchanged in its composition. In this case, it is in principle possible and according to the invention also preferred to introduce the 1,2-propanediol containing phase P₁ into the subsequent reactor R₂ together with the preferably gaseous hydrogen phase H₁. It is however also conceivable first to separate off the preferably liquid 1,2-propanediol containing phase P₁ from the hydrogen phase H₁, this separating-off taking place preferably in separating devices known to a person skilled in the art, such as for example a separator. Separating devices suitable for this purpose are described for example in Chapter 4.2.1 in “Grundoperationen Chemischer Verfahrenstechnik” [Basic Operations of Chemical Processed Engineering] , Wilhelm R. A. Vauck and Hermann A. Muller, Wiley-VCH-Verlag, 11^{th} edition. Suitable separating devices include in particular chamber separators, impingement separators and centrifugal separators. After separating-off of the liquid 1,2-propanediol containing phase from the hydrogen phase H₁, the preferably liquid 1,2-propanediol containing phase P₁ can then be introduced into the subsequent reactor.

In addition to the preferably liquid 1,2-propanediol containing phase P₁, which contains in addition to 1,2-propanediol also not yet reacted glycerol and by-products formed in the reactor R₁, the hydrogen required for hydrogenation is also introduced into the subsequent reactor R₂. This hydrogen is preferably the hydrogen phase H₁ which issues from the preceding reactor R₁ and was optionally separated off beforehand from the 1,2-propanediol containing phase P₁. Also conceivable however is the use of fresh, preferably cold hydrogen, i.e. hydrogen gas which has not previously passed through the reactor R₁, or else the use of a mixture of the hydrogen phase H₁ and fresh hydrogen gas.

In principle, the hydrogen gas can be introduced into the reactor R₂ together with the 1,2-propanediol containing phase P₁ or else separately from the 1,2-propanediol containing phase P₁, wherein the joint or separate introduction of the 1,2-propanediol containing phase P₁ and the hydrogen gas into the subsequent reactor R₂ can in principle take place in the same manner in which the glycerol phase P_{glycerol} and the hydrogen gas are also introduced into the first reactor R₁. According to the invention, it is preferred that the 1,2-propanediol containing phase P₁ and the hydrogen gas are introduced jointly, the reactor R₂ being arranged perpendicularly, from above into the reactor.

In the reactor R₂, the glycerol which is obtained in the 1,2-propanediol containing phase P₁ and has not yet reacted in the first reactor is now further reacted to form 1,2-propanediol, and the reaction taking place preferably under the pressure and temperature conditions mentioned in relation to the reactor R₁. On issuing from this reactor, a preferably liquid 1,2-propanediol containing phase P₂ and a preferably gaseous hydrogen phase H₂ are obtained, which can optionally (where i>2) be introduced jointly or separately into a further reactor R₃, wherein the separation can again take place preferably by means of the above-mentioned separating devices (also conceivable, as previously stated in relation to the reactor R₃, is the introduction of fresh hydrogen gas or a mixture of fresh hydrogen gas and the hydrogen phase H₂ into the reactor R₃). According to the invention, it is in this connection furthermore preferred that the glycerol is reacted in the second reactor R₂ preferably to 20 to 60 mol % , preferably 30 to 50 mol %, most preferably 35-40 mol %, based in each case on the total amount of glycerol introduced into the reactor R₁. The total reaction if only two successive reactors (i=2) are used is thus preferably approximately 85 mol % (approximately 50 mol % in the first reactor and approximately 35 mol % in the second reactor).

According to the most preferred embodiment of the process according to the invention, two successive reactors are used (i=2). According to a further embodiment, three successive reactors are used in the process according to the invention (i=3). According to a further embodiment, four successive reactors are used in the process according to the invention (i=4). According to another embodiment, at least five, at least six, at least seven, at least eight, at least nine or at least ten successive reactors are used in the process according to the invention (i>5, i>6, i>7, i>8, i>9 or i>10). According to the invention, it is therefore preferred if i is an integer from the range of from 2 to 10, particularly preferably from the range of from 2 to 5.

As described at the outset, it is preferred in the process according to the invention that each 1,2-propanediol containing phase Pₙ is cooled
i) within each reactor Rₙ,
ii) in a region between the reactor Rₙ and Rₙ₊₁, or
iii) within each reactor Rₙ and in a region between the reactor Rₙ and the reactor Rₙ₊₁.

If the 1,2-propanediol containing phases are cooled according to alternative i) inside the reactors, the cooling is preferably carried out
I) by passing cold hydrogen gas into the reactor Rₙ, or
II) by a cooling device inside the reactor Rₙ, or
III) by passing cold hydrogen gas into the reactor Rₙ and by a cooling device inside the reactor Rₙ.

The introduction of hydrogen gas according to alternative I) is preferably carried out in such a way that the hydrogen gas is introduced through lances or tubes, which are arranged parallel to the longitudinal axis inside the tubular reactor which is preferred in accordance with the invention and have spaced holes, and passes into the catalyst bed via these holes. In this case, it is particularly preferred that the holes are located in the lower two thirds of the reactor, particularly preferably in the lower half of the perpendicularly arranged reactor (insofar as the hydrogen used for reduction (circulating gas or fresh hydrogen) and the glycerol are fed in from above). Furthermore, it is in this connection preferred that the diameter of the holes in the lance or in the tube is larger, the deeper the point at which the hole is located projects into the catalyst fixed bed. Used as hydrogen, which is used for cooling (=“quench gas”), is preferably a portion of the circulating gas that was obtained at the end of the last reactor Rᵢ after cooling and separating-off from the 1,2-propanediol containing phase Pᵢ. Also conceivable is the use of fresh hydrogen gas as the quench gas or else a mixture of fresh hydrogen gas and quench gas. The temperature of the hydrogen quench gas introduced inside the reactor Rₙ for cooling the 1,2-propanediol containing phase is preferably in a range of from 40 to 100° C., particularly preferably in a range of from 60 to 80° C.

If the 1,2-propanediol containing phase Pₙ is cooled inside the reactor Rₙ by means of a cooling device according to alternative II), attached inside the reactor are cooling coils, heat exchange plates through which cooling liquids flow and the like, by means of which the 1,2-propanediol containing phase can be cooled inside the reactor. In this case too, it can be advantageous to provide the cooling devices in the lower two thirds of the reactor, particularly preferably in the lower half of the perpendicularly arranged reactor (insofar as the hydrogen used for reduction (circulating gas or fresh hydrogen) and the glycerol are fed in from above).

If the 1,2-propanediol containing phases are cooled according to alternative ii) in a region between the reactor Rₙ and Rₙ₊₁, it is preferred if a cooling region K_{n/n+1} is arranged between the reactor Rₙ and the reactor Rₙ₊₁, through which the 1,2-propanediol containing phase P. issuing from the reactor Rₙ and optionally also the hydrogen phase Hₙ issuing from the reactor Rₙ, insofar as said hydrogen phase is also introduced into the subsequent reactor Rₙ₊₁, are cooled before they enter the reactor Rₙ₊₁. In this case, it has been found that the cooling of at least one of these two phases, particularly preferably the 1,2-propanediol containing phase Pₙ, even more preferably the 1,2-propanediol containing phase Pₙ and the hydrogen phase Hₙ, insofar as said hydrogen phase is also introduced into the subsequent reactor Rₙ₊₁, before they enter the subsequent reactor advantageously influences the selectivity of the hydrogenation of glycerol. As a result of this cooling of at least one of these phases, preferably of both phases, noticeably fewer by-products are formed.

The cooling region K_{n/n+1} can in this case comprise all devices which are known to a person skilled in the art and by means of which liquid or gaseous fluids or mixtures of liquid and gaseous fluids can be cooled. Examples include in this connection, in particular, heat exchangers such as for example coiled tubular heat exchangers, double-tube heat exchangers, shell-and-tube heat exchangers, lamella bundle heat exchangers, fin-tube heat exchangers or plate-type heat exchangers. Heat exchangers of this type are described in Chapter 8.1.5 in “Grundoperationen Chemischer Verfahrenstechnik” [Basic Operations of Chemical Process Engineering] , Wilhelm R. A. Vauck and Hermann A. Müller, Wiley-VCH-Verlag, 11^{th} edition. A solid, liquid or gaseous refrigerant is generally used for cooling in devices of this type. As refrigerants in particular water, heat transfer liquids such as Marlotherm® or molten salts can be used. In many cases, it is advantageous to pass these refrigerants through the heat exchanger in countercurrent, for example, to the 1,2-propanediol containing phase to be cooled. According to one particular embodiment of the process according to the invention, the refrigerant used can also be hydrogen gas, the glycerol phase P_{glycerol} or else a mixture of hydrogen gas and the glycerol phase P_{glylcerol}. That is to say, in this way, the educts introduced into the reactor R₁ or the educt mixture introduced into the reactor R₁ can already be preheated in an advantageous manner.

In relation to the cooling region K_{n/n+1}, it is furthermore preferred that this cooling region K_{n/n+1} causes cooling by 10 to 70° C., particularly preferably by 20 to 60° C., even more preferably by 30 to 50° C. of the 1,2-propanediol containing phase Pₙ issuing from the reactor Rₙ and/or, preferably and, of the hydrogen phase Hₙ issuing from the reactor Rₙ, insofar as said hydrogen phase is introduced into the subsequent reactor Rₙ₊₁, before they enter the reactor Rₙ₊₁.

Furthermore, the 1,2-propanediol containing phases, the hydrogen phases or the mixtures of these phases can in principle be cooled by means of the cooling regions K_{n/n+1} in such a way that the respective liquid 1,2-propanediol containing phase Pₙ is cooled together with the gaseous hydrogen phase Hₙ issuing from the same reactor in the cooling region K_{n/n+1}; it is however also conceivable first to separate the liquid 1,2-propanediol containing phase Pₙ from the gaseous hydrogen phase Hₙ, as described hereinbefore, by means of a separator and then to subject the liquid 1,2-propanediol containing phase, the gaseous hydrogen phase or else both phases separately from each other to cooling in a common cooling region or in separate cooling regions. However, common cooling of the 1,2-propanediol containing phase Pₙ and the hydrogen phase Hₙ in the cooling region K_{n/n+1} is preferred.

Furthermore, it can according to the invention be advantageous if 1,2-propanediol is purified from at least one of the 1,2-propanediol containing phases Pₙ, n being an integer from the range of from 2 to i-1, preferably from the 1,2-propanediol containing phase Pᵢ. For this purpose, preferably in a high-pressure separator arranged after the reactor from which the 1,2-propanediol containing phase to be purified issues, preferably after the reactor Rᵢ the preferably liquid 1,2-propanediol containing phase is separated off, after prior cooling, from the preferably gaseous hydrogen phase and subsequently stress-relieved. From the 1,2-propanediol containing phase thus obtained, the 1,2-propanediol can then optionally be further purified, this purifying being carried out using purification processes known to a person skilled in the art, for example by simple distillation, by rectification, by crystallization or by extraction, particularly preferably however by simple distillation or by rectification.

Furthermore, it can according to the invention be advantageous to use for the reactors R₁ to Rᵢ reactors which differ in size, in particular with regard to their reactor volume, it being particularly preferred to select a cascade arrangement in which the volume of the reactors R₂ to Rᵢ, in particular the average volume of the reactors R₂ to Rᵢ, is greater by at least 30%, preferably by at least 50%, even more preferably by at least 100% and most preferably by at least 150% than the volume of the first reactor R₁. The volume of the reactor R₁, which is smaller than that of the subsequent reactors, enables in particular allowance to be made for the circumstance that per unit of volume the highest exothermy is to be expected in this reactor on reaction of the glycerol phase P_{glycerol} used therein with hydrogen.

According to a preferred embodiment of the process according to the invention, two reactors (i=2) are used, wherein
a glycerol phase P_{glycerol} and hydrogen gas are jointly heated in a heater to a temperature in a range of from approximately 180 to 220° C. and are introduced into the first reactor, in the reactor R₁ hydrogen additionally being introduced as the quench gas into the catalyst fixed bed for cooling via a tube which has holes and is passed into the reactor,
the 1,2-propanediol containing phase P₁ issuing from the reactor R₁ is cooled together with the hydrogen phase H₁ issuing from the reactor R₁ to a temperature in a range of from 180 to 220° C. in a cooling region K_{1/2} arranged between the first and the second reactor,
the cooled 1,2-propanediol containing phase is subsequently introduced into the reactor R₂ together with the hydrogen phase H₁, in the reactor R₂ hydrogen also additionally being introduced as the quench gas into the catalyst fixed bed for cooling via a tube which has holes and is passed into the reactor,
the 1,2-propanediol containing phase P₂ issuing from the reactor R₂ is subsequently cooled, optionally together with the hydrogen phase H₂ also issuing from the reactor R₂, separated in a further separator from the hydrogen phase H₂, stress-relieved and then optionally supplied to a further purification device in which the 1,2-propanediol containing phase is further purified.

According to a further preferred embodiment of the process according to the invention, the preferably liquid 1,2-propanediol containing phase Pᵢ issuing from the reactor Rᵢ contains, after separation of the hydrogen phase Hᵢ glycerol contamination in a range of from 0.01 to 20% by weight, or from 0.1 to 15% by weight, or from 1 to 10% by weight, based in each case on the total amount of the phase Pᵢ.

According to a further preferred embodiment, the ratio of glycerol to 1,2-propanediol in the preferably liquid 1,2-propanediol containing phase Pᵢ issuing from the reactor Rᵢ is, after separation of the hydrogen phase Hᵢ in a range of from 1:3 to 1:8.

A contribution to achieving the object mentioned at the outset is provided also by a 1,2-propanediol containing phase Pᵢ or else purified 1,2-propanediol which is obtainable using the process according to the invention described hereinbefore.

A further contribution to achieving the object mentioned at the outset is provided by a device for preparing 1,2-propanediol, containing at least i fluidically interconnected reactors R₁ to Rᵢ which have a hydrogenation catalyst and are preferably a tubular reactor, wherein
a) between at least one pair of adjacent reactors Rₙ and Rₙ₊₁, n being an integer from the range of from 1 to i, a cooling region K_{n/n+1} is provided, through which the 1,2-propanediol containing phase Pₙ issuing from the reactor Rₙ is cooled, optionally together with hydrogen, prior to entering the reactor Rₙ₊₁,
b) within at least one reactor Rₙ a cooling device is provided, or
c) between at least one pair of adjacent reactors Rₙ and Rₙ₊₁, n being an integer from the range of from 1 to i, a cooling region K_{n/n+1} is provided, through which the 1,2-propanediol containing phase Pₙ issuing from the reactor Rₙ is cooled, optionally together with hydrogen, prior to entering the reactor Rₙ₊₁, and a cooling device is provided in at least one reactor Rₙ.

The term “fluidically” means in this case that the reactors R₁ to R, are flow-connected to one another so as to allow both liquids and gases to be passed from one reactor, optionally through the cooling region connected to this reactor, into the subsequent reactor.

Preferred as reactors, hydrogenation catalysts and cooling regions are in this case those reactors, hydrogenation catalysts and cooling regions which were mentioned at the outset in relation to the process according to the invention for preparing 1,2-propanediol as preferred reactors, hydrogenation catalysts and cooling regions, respectively. In particular, it is preferred that at least one of the reactors R₁ to Rᵢ, preferably all these reactors, contains a heterogeneous, copper or chromium-containing catalyst, in particular a heterogeneous copper chromite catalyst.

Examples of cooling devices which can be provided inside the reactor according to variants b) and c) include on the one hand those cooling devices which were described hereinbefore in relation to variant II) of the process according to the invention as being a suitable cooling device. Conceivable and according to the invention particularly preferred as a cooling device is however also the tube which was described at the outset in relation to the process according to the invention and has holes and via which hydrogen, as the quench gas, can be introduced directly into the catalyst bed inside the reactor. Also conceivable is however a supply of quench gas from below or through lances which enter the reactor laterally and can be regulated individually by external valves with regard to the amount of hydrogen.

According to a preferred embodiment of the device according to the invention, said device further comprises a fat cleaver which is fluidically connected to at least one of the reactors R₁ to Rᵢ, preferably to the reactor R₁, so that the glycerol-containing reaction phase which is obtained in the fat cleaver can be introduced, optionally after prior cooling and/or purifying, as a glycerol phase P_{glycerol} into at least one of the reactors R₁ to Rᵢ preferably into the reactor R₁.

A contribution to achieving the objects mentioned at the outset is provided also by a process for preparing 1,2-propanediol by hydrogenation of glycerol by means of hydrogen gas, using the device described hereinbefore.

A further contribution to achieving the objects mentioned at the outset is provided by a process for preparing a compound having at least one ether group, at least one ester group, at least one amino group, at least one urethane group, or at least two thereof, preferably having at least one ester group, including the process steps:
i) providing a 1,2-propanediol containing phase Pᵢ using the process according to the invention described hereinbefore, the 1,2-propanediol optionally having been purified from the 1,2-propanediol containing phase Pᵢ,
ii) reacting the 1,2-propanediol contained in the 1,2-propanediol containing phase Pᵢ or the purified 1,2-propanediol with a compound having at least one active hydrogen atom, at least one epoxide group, at least one ester group or at least one isocyanate group.

According to a particularly preferred embodiment of the process according to the invention for preparing a compound having at least one ether group, at least one ester group, at least one amino group, at least one urethane group, or at least two thereof, preferably having at least one ester group, in process step i) a 1,2-propanediol containing phase Pᵢ is provided, which was obtained as the 1,2-propanediol containing phase P₂ in the process according to the invention for preparing 1,2-propanediol wherein i=2.

According to a further preferred embodiment, the i)-1,2-propanediol containing phase Pᵢ provided in process step i) and prepared using the process according to the invention contains a glycerol contamination in a range of from 0.01 to 20% by weight, or from 0.1 to 15% by weight, or from 1 to 10% by weight, based in each case on the total amount of the phase Pᵢ, and

the ii)-1,2-propanediol containing phase Pᵢ used in process step ii) contains at least 70 up to 100% by weight of the glycerol contamination of the i)-1,2-propanediol containing phase P, provided in process step i).

In accordance with the foregoing, the i)-1,2-propanediol containing phase Pᵢ obtained in process step i) can be used in process step ii) as the ii)-1,2-propanediol containing phase Pᵢ substantially without intermediate treatment or purification steps.

In this case, it is generally not necessary to subject the i)-1,2-propanediol containing phase to thermal separation processes in order to separate off all of the impurities contained. Nevertheless, it is possible, for example, as stated above, or by applying a suitable vacuum, to remove a portion of the glycerol contamination, or else portions of further contaminations such as for example monoalcohols. Equally, it may prove advantageous to separate off at least solids, such as for example catalysts, this being carried out preferably using non-thermal separation processes such as filtration, sedimentation or centrifugation.

According to a further preferred embodiment, the ratio of glycerol to 1,2-propanediol in the 1,2-propanediol containing phase Pᵢ provided in process step i) is in a range of from 1:3 to 1:99, preferably from 1:3 to 1:50, or from 1:2.5 to 1:8.

A compound having at least one active hydrogen atom in this case preferably means a compound having at least one hydrogen atom which is bound to an atom different from carbon, preferably to an oxygen atom, a nitrogen atom or a sulphur atom, particularly preferably to an oxygen atom or a nitrogen atom and most preferably to an oxygen atom. These compounds having at least one active oxygen atom therefore have preferably an OH group, a COOH group, an NH₂ group, an NRH group (wherein R is a further organic radical, such as for example an alkyl or alkenyl group) or an SH group.

If the compound having at least one active hydrogen atom is a compound having at least one hydroxyl group, then after reaction of this compound with the 1,2-propanediol containing phase Pᵢ or of the purified 1,2-propanediol, a compound having at least one ether group is obtained. Depending on the molar ratio at which the compound having at least one hydroxyl group is reacted with the 1,2-propanediol contained in the 1,2-propanediol containing phase Pᵢ or with the purified 1,2-propanediol, a polyether compound can also be obtained.

If the compound having at least one active hydrogen atom is a compound having at least one carboxylic acid group, then after reaction of this compound with the 1,2-propanediol containing phase Pᵢ or with the purified 1,2-propanediol, a compound having at least one ester group is obtained. Depending on the molar ratio at which the compound having at least one carboxyl group is reacted with the 1,2-propanediol contained in the 1,2-propanediol containing phase Pᵢ or with the purified 1,2-propanediol, an ester-polyether compound can be obtained.

If the compound having at least one active hydrogen atom is a compound having at least one amino group, then after reaction of this compound with the 1,2-propanediol containing phase Pᵢ or with the purified 1,2-propanediol, a compound having at least one amino group and at least one hydroxyl group is obtained. Depending on the molar ratio at which the compound having at least one amino group is reacted with the 1,2-propanediol contained in the 1,2-propanediol containing phase Pᵢ or with the purified 1,2-propanediol, an aminopolyether compound can also be obtained.

If a compound having at least one epoxide group is used in process step ii), ether or polyether can also be obtained as a result of the reaction of this compound with the 1,2-propanediol containing phase Pᵢ or with the purified 1,2-propanediol.

If a compound having at least one isocyanate group is used in process step ii), urethanes or polyurethanes can be obtained as a result of the reaction of this compound with the 1,2-propanediol containing phase Pᵢ or with the purified 1,2-propanediol.

Particularly preferred according to the invention is the use of a compound having at least one carboxylic acid group in process step ii). In this case, the compound having at least one carboxylic acid group can be a monocarboxylic acid, a dicarboxylic acid or a tricarboxylic acid, monocarboxylic and dicarboxylic acids being particularly preferred and monocarboxylic acids, in particular fatty acids, most preferred. Furthermore, it is preferred that the compound having at least one carboxylic acid has 5 to 30, particularly preferably 10 to 25 and most preferably 15 to 20 carbon atoms per molecule. In this connection, it is particularly preferred that the compound having at least one carboxylic acid group is a C₅ to C₃₀ monocarboxylic acid, additionally preferably a C₁₀ to C₂₅ monocarboxylic acid and most preferably a C₁₅ to C₂₀ monocarboxylic acid, the above-mentioned monocarboxylic acids being saturated monocarboxylic acids, such as for example caproic acid, heptanoic acid, caprylic acid, pelargonic acid, capric acid, lauric acid, myristic acid, fish oil, palmitic acid, margaric acid, stearic acid, arachic acid, behenic acid, lignoceric acid or cerotic acid, singly unsaturated monocarboxylic acids such as for example undecylenic acid, oleic acid, elaidic acid, vaccenic acid, eicosenoic acid, cetoleic acid, erucic acid or nervonic acid, or multiply unsaturated monocarboxylic acids, such as for example linoleic acid, linolenic acid, arachidonic acid, timnodonic acid, clupanodonic acid or cervonic acid.

The above-mentioned fatty acids can be obtained for example from vegetable oils, hydrogenated vegetable oils, marine oils and animal fats and oils. Preferred vegetable oils include corn oil, canola oil, olive oil, cotton seed oil, soya oil, cocoa oil, palm kernel oil, sunflower oil, rapeseed oil, in particular high-erucic acid rapeseed oil, partially or completely hydrogenated soya oil, partially or completely hydrogenated canola oil, partially or completely hydrogenated sunflower oil, partially or completely hydrogenated high-erucic acid rapeseed oil, partially or completely hydrogenated cotton seed oil, palm oil or palm stearin.

In the case of a dicarboxylic acid in particular compounds selected from the group consisting of phthalic anhydride; isophthalic acid; terephthalic acid; tetrahydrophthalic anhydride; hexahydrophthalic anhydride; naphthalinedicarboxylic acid; 4,4′-biphenyldicarboxylic acid; diphenylmethane-4,4′-dicarboxylic acid; succinic acid; fumaric acid; adipic acid; sebacic acid; azelaic acid and maleic anhydride can be used, of which terephthalic acid is most preferred.

The compound having at least one carboxylic acid group is reacted in process step ii) of the process according to the invention as the acid component with 1,2-propanediol as the alcohol component so as to obtain an ester. In this case, it is however in principle also possible to use 1,2-propanediol not as the single alcohol component, but rather additionally to use at least one further alcohol component, so that a compound having at least two different ester groups is obtained. This at least one further alcohol component can be a tetrahydric or polyhydric alcohol, such as for example diglycerol; triglycerol; polyglycerol; pentaerythritol; dipentaerythritol or sorbitol; or else a trihydric; dihydric or monohydric alcohol; such as for example trimethylolpropane; trimethylolethane; a dihydric alcohol such as for example ethylene glycol; diethylene glycol; triethylene glycol; 1,3-propanediol; 1,4-butanediol; 1,5-pentanediol; neopentyl glycol; 1,6-hexanediol; 1,4-dimethylolcyclohexane; methanol; ethanol; 1-propanol or 2-propanol. Insofar as at least one further alcohol component is used, it is however preferred that the proportion formed by this further alcohol component of the total amount of 1,2-propanediol and further alcohol component is at most 50% by weight, particularly preferably at most 25% by weight, additionally preferably at most 10% by weight and most preferably at most 5% by weight.

According to one particular embodiment of the process according to the invention, the 1,2-propanediol containing phase P₂ obtained in process step i) without prior purifying (optionally however after separating-off of the hydrogen and stress relief) which was obtained in a process wherein i=2 is reacted with a fatty acid so as to obtain a fatty acid ester.

Furthermore, it is preferred that the fatty acid is reacted with the alcohol component, i.e. with the 1,2-propanediol contained in the 1,2-propanediol containing phase Pᵢ or with a mixture of the 1,2-propanediol and at least one further alcohol in the presence of an esterification catalyst. The esterification catalysts used can be acids, such as for example sulphuric acid or p-toluenesulphonic acid, or metals and the compounds thereof. Suitable are for example tin, titanium, zirconium which are used as finely divided metals or expediently in the form of their salts, oxides or soluble organic compounds. The metal catalysts are, in contrast to protonic acids, high-temperature catalysts which generally achieve their full activity only at temperatures above 180° C. They are however preferred according to the invention, because they supply fewer by-products, such as for example olefins, than proton catalysis. Esterification catalysts which are particularly preferred in accordance with the invention are one or more divalent tin compounds or tin compounds or elemental tin which can be reacted with the educts to form divalent tin compounds. For example, the catalyst used can be tin, tin (II) chloride, tin (II) sulphate, tin (II) alcoholates or tin (II) salts of organic acids, in particular of monocarboxylic and dicarboxylic acids. Particularly preferred tin catalysts are tin (II) oxalate and tin (II) benzoate.

The esterification reaction can be carried out using the process known to a person skilled in the art. In this case, it can be particularly advantageous to remove the water formed during the reaction from the reaction mixture, this removal of the water being carried out preferably by distillation, optionally by distillation with excess 1,2-propanediol. 1,2-Propanediol which has not reacted after carrying out the esterification reaction can also be removed from the reaction mixture, this removal of the 1,2-propanediol also being carried out preferably by means of distillation. Furthermore, after completion of the esterification reaction, in particular after the separating-off of non-reacted 1,2-propanediol, the catalyst remaining in the reaction mixture can be separated off, optionally after treatment with a base, by filtration or by centrifuging.

Furthermore, it is preferred to carry out the esterification reaction at a temperature in a range of from 50 to 300° C., particularly preferably in a range of from 100 to 250° C. and most preferably in a range of from 150 to 200° C. The optimum temperatures depend on the alcohol(s) used, the progress of the reaction, the type of catalyst and the catalyst concentration. They can easily be determined for each individual case by carrying out tests. Elevated temperatures increase the reaction speeds and promote secondary reactions, such as for example the splitting-off of water from alcohols or the formation of colored by-products. The desired temperature or the desired temperature range can be set by way of the pressure in the reaction vessel (slight excess pressure, normal pressure or optionally reduced pressure).

According to another particular embodiment of the process according to the invention, the 1,2-propanediol containing phase Pᵢ obtained in process step i), preferably the 1,2-propanediol containing phase P₂ obtained in a process wherein i=2, or else 1,2-propanediol purified from this phase is reacted with a dicarboxylic or tricarboxylic acid and a fatty acid so as to obtain an alkyd resin.

Alkyd resins are synthetic, highly hydrophobic polymers obtained by condensation of diols (in the present case of 1,2-propanediol) with polybasic acids with the addition of organic oils or fatty acids (to modify the properties of the resin) and optionally further, polyhydric alcohols, in particular glycerol or pentaerythritol. In this case, it is particularly preferred that the compound having at least one carboxylic acid group is a dibasic acid which is preferably selected from the group consisting of phthalic anhydride; isophthalic acid; terephthalic acid; tetrahydrophthalic anhydride; hexahydrophthalic anhydride; naphthalinedicarboxylic acid; 4,4′-biphenyldicarboxylic acid; diphenylmethane-4,4′-dicarboxylic acid; succinic acid; fumaric acid; adipic acid; azelaic acid; sebacic acid and maleic anhydride, terephthalic acid being most preferred.

Examples of organic oleic or fatty acids include in particular tallows, canola oil, rape oil, sunflower oil, palm oil, which can optionally be present also in hardened or semi-hardened form, soya bean oil, thistle oil, linseed oil, tall oil, coconut oil, palm kernel oil, castor oil, dehydrogenated castor oil, fish oil and tung oil. Particularly preferred are drying oils or semidrying oils having iodine values of at least 50; inter alia soya bean oil, tall oil and in particular also tallow are advantageous. Examples of fatty acids used both for preparing the alkyd resins and for preparing the fatty acid esters include in particular those of soya bean oil, thistle oil, linseed oil, tall oil, coconut oil, palm kernel oil, castor oil, dehydrogenated castor oil, fish oil and tung oil. Of these fatty acids, those of drying oils or semidrying oils having iodine values of at least 100, inter alia those of soya bean oil and tall oil, are preferred.

The preparation of alkyd resins may be inferred for example from WO-A-01/62823, the disclosure of which with regard to the preparation of alkyd resins is incorporated herein by reference.

Also conceivable is the use of a compound having at least one hydroxy group in process step ii). In this case, the compound having at least one hydroxy group can be a monool, a diol, a triol or an alcohol having more than three OH groups. Particularly preferred compounds having at least one OH group are however fatty acid alcohols which were obtained by reduction of fatty acid esters, for example with sodium in a Bouveault-Blanc reaction. Fatty alcohols which are suitable in this connection are for example hexanol, octanol, decanol, dodecanol, tetradecanol, hexadecanol, heptadecanol, octadecanol, eicosanol, behenyl alcohol, delta-9-cis-hexadecenol, delta-9-octadecenol, trans-delta-9-octadecenol, cis-delta-11-octadecenol or octadecatrienol.

The preparation of ethers from fatty alcohols and 1,2-propanediol, in particular the preparation of polyethers by the polyproxylation of fatty alcohols with 1,2-propanediol is carried out also preferably by means of suitable catalysts, such as calcium and strontium hydroxides, alkoxides or phenoxides (EP-A-0 092 256), calcium alkoxides (EP-A-0 091 146), barium hydroxide (EP-B-0 115 083), basic magnesium compounds, such as for example alkoxides (EP-A-0 082 569), magnesium and calcium fatty acid salts (EP-A-0 085 167), antimony pentachloride (DE-A-26 32 953), aluminum isopropylate/sulphuric acid (EP-A-0 228 121), zinc, aluminum and other metal-containing oxo compounds (EP-A-0 180 266) or aluminum alcohols/phosphoric acids (U.S. Pat. No. 4,721,817). More precise information for preparing polypropxylated fatty alcohols can be inferred inter alia also from EP-A-0 361 083, the disclosure of which with regard to the preparation of polyethers from 1,2-propanediol and fatty alcohols is incorporated herein by reference.

Also conceivable is the use of a compound having at least one amino group in process step ii). In this case, the compound having at least one amino group can in particular be a fatty amine which can be obtained from triglycerides by treatment with ammonia and subsequent hydrogenation. The propoxylation of amines with 1,2-propanediol is also described in EP-A-0 361 083, the disclosure of which is incorporated herein by reference.

When use is made of a compound having at least one epoxide group in process step ii), the 1,2-propanediol containing phase Pᵢ, preferably the 1,2-propanediol containing phase P₂ obtained in a process wherein i=2, or 1,2-propanediol purified from this phase is reacted with compounds such as for example ethylene oxide, propylene oxide, ethylene diglycidyl ether, propylene diglycidyl ether, diethylene diglycidyl ether, dipropylene diglycidyl ether, triethylene diglycidyl ether, tripropylene diglycidyl ether, tetraethylene diglycidyl ether, tetrapropylene diglycidyl ether or polyethylene diglycidyl ethers or polypropylene diglycidyl ethers having a still higher molecular weight, wherein polyethers can also be obtained.

When use is made of a compound having at least one ester group in process step ii), the reaction with 1,2-propanediol results in transesterification, esters of the above-mentioned mono fatty acids preferably being used as the ester having at least one ester group.

When use is made of a compound having at least one isocyanate group in process step ii), the 1,2-propanediol containing phase Pᵢ, preferably the 1,2-propanediol containing phase P₂ obtained in a process wherein i=2, or 1,2-propanediol purified from this phase is reacted preferably with diisocyanates, such as for example hexamethylene diisocyanate (HDI), isophorone diisocyanate (IPDI), toluylene diisocyanate (TDI), diphenylmethane diisocyanate (MDI) or mixtures of diphenylmethane diisocyanate and polymethylene polyphenylene polyisocyanates, in the presence of suitable catalysts. A suitable process for preparing polyurethanes is for example that in DE-A-10 2004 041 299, the disclosure of the preparation of polyurethanes from diols and polyisocyanates is incorporated herein by reference.

According to a particularly preferred embodiment of the process according to the invention, the 1,2-propanediol containing phase P, obtained in process step i), preferably the 1,2-propanediol containing phase P₂ obtained in a process wherein i=2, is not purified, preferably is at least not purified by thermal separation processes, such as for example distillation or rectification, prior to the reaction in process step ii). The 1,2-propanediol containing phase is therefore supplied directly to the reaction with a compound having at least one active hydrogen atom, at least one epoxide group, at least one ester group or at least one isocyanate group. Nevertheless, it can prove advantageous to separate off from the 1,2-propanediol containing phase at least solids, such as for example catalysts, this separating-off being carried out preferably by way of non-thermal separation processes, i.e. for example by filtration, sedimentation or centrifugation.

A contribution to achieving the objects mentioned at the outset is provided also by a compound having at least one ether group, at least one ester group or at least one urethane group, preferably having at least one ester group which is obtainable, preferably was obtained, by way of the process according to the invention described hereinbefore. In this case, it is particularly preferred that this compound is a mono fatty acid ester which was obtained by reacting the 1,2-propanediol containing phase P. obtained in process step i), preferably the 1,2-propanediol containing phase P₁ obtained in a process wherein i=2, or 1,2-propanediol purified from this phase, with a fatty acid, preferably with a mono fatty acid.

The present invention will now be described in greater detail with reference to non-limiting figures and examples.

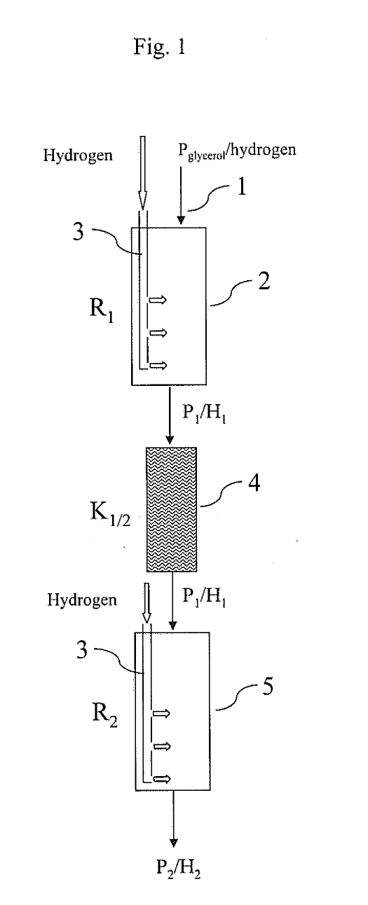

FIG. 1 shows a particular configuration of the process according to the invention or the device according to the invention, in which two reactors are provided with an interposed cooling region.

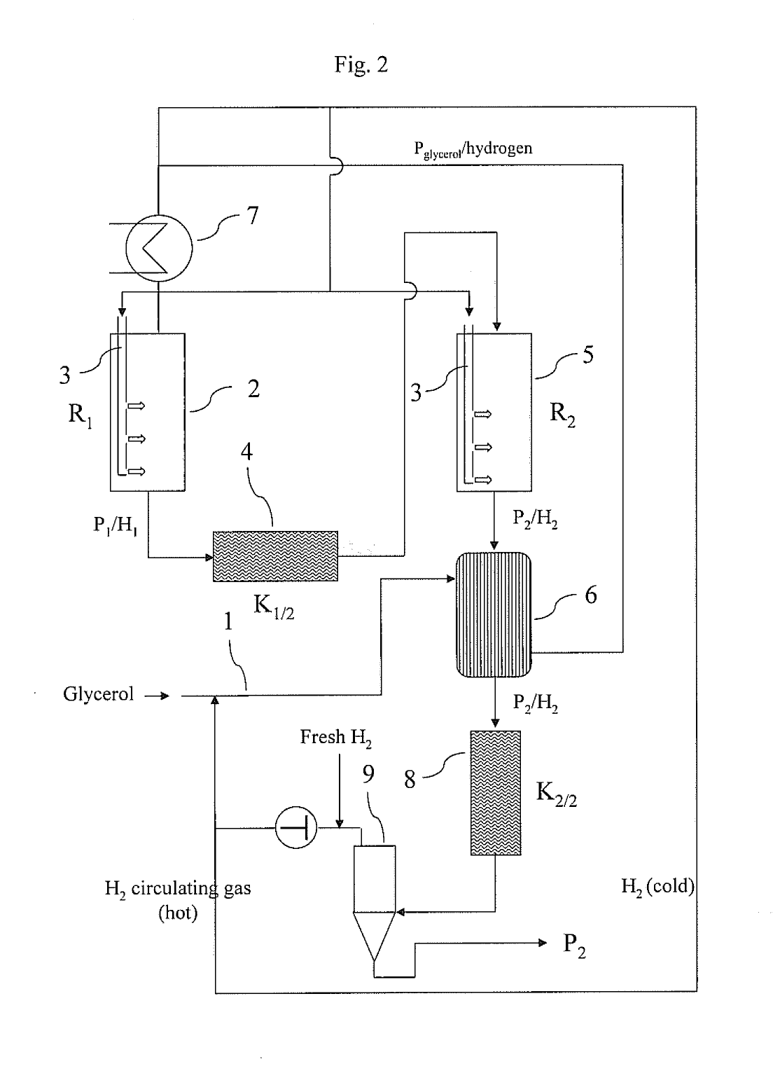

FIG. 2 shows a further particular configuration of the process according to the invention or the device according to the invention with two successive tubular reactors in which the hydrogen gas issuing from the last reactor is returned as a quench gas to the reactors R₁ and R₂ and as a gas for hydrogenation to the reactor R₁.

According to FIG. 1, hydrogen gas and a glycerol phase P_{glycerol} are introduced into the first reactor R₁ (2) via a feed line 1. This reactor R₁ (2) is preferably a tubular reactor containing a catalyst charge based on a Cu chromite catalyst. Preferably, the glycerol phase P_{glycerol} is passed through the reactor R₁ in trickle bed operation in parallel or countercurrent flow with hydrogen. Within the reactor R₁, the 1,2-propanediol containing phase P₁ is cooled by hydrogen quench gas which passes into the reactor R₁ (2) via a tube (3) which has holes. Alternatively, inside the reactor R₁ (2), cooling can be carried out also by other suitable cooling devices, such as for example by cooling coils and the like. At the outlet of the reactor R₁ (2), a liquid 1,2-propanediol containing phase P₁ and a gaseous hydrogen phase H₁ are obtained, which are then jointly passed through a cooling region K_{1/2} (4). It is however also conceivable first to separate the 1,2-propanediol containing phase Pᵢ in a separator from the hydrogen phase H₁ and then to pass the 1,2-propanediol containing phase P₁ separated from the hydrogen phase H₁ through the cooling region K_{1/2}. The cooling region is a cooling device, such as for example a heat exchanger, in particular a coiled tubular heat exchanger, a double-tube heat exchanger, a shell-and-tube heat exchanger, a lamella bundle heat exchanger, a fin-tube heat exchanger or a plate-type heat exchanger. After passing through the cooling region K_{1/2}, the cooled 1,2-propanediol containing phase P₁ is introduced together with the cooled hydrogen phase Hᵢ into the second reactor R₂ (5) which is also preferably a tubular reactor having a catalyst fixed bed containing a Cu chromite catalyst. In the reactor R₂ too, the 1,2-propanediol containing phase P₂ formed therein is cooled by hydrogen quench gas which also passes into the interior of the reactor via a tube (3) provided with holes. The 1,2-propanediol containing phase P₁ is also passed into the reactor R₂ (5), preferably in trickle bed operation in parallel or countercurrent flow with hydrogen. At the outlet of the reactor R₂, a preferably liquid 1,2-propanediol containing phase P₂ and a preferably gaseous hydrogen phase H₂ are obtained.

FIG. 2 shows a particular embodiment of the procedure described in FIG. 1. According to FIG. 2, the mixture of the glycerol phase P_{glycerol} and the hydrogen gas, which is used as an educt composition, is first passed through a heat exchanger 6 and in this way already preheated. Then, the educt composition is heated by means of a heating device 7 to a temperature in a range of from 180 to 220° C. and introduced into the reactor (2) where the glycerol is reacted to approximately 50 mol %. After leaving the reactor Rᵢ (1), the 1,2-propanediol containing phase P₁ is passed together with the hydrogen phase H₁ through a cooler K_{1/2} (4) which cools the composition to a temperature in a range of from 180 to 220° C. Then, the composition is introduced into the reactor R₂ (5) in which the remaining glycerol is reacted to approximately 35 mol %. At the outlet of the reactor R₂ (5), a 1,2-propanediol containing phase P₂ and a hydrogen phase H₂ are obtained, which are first passed jointly through the heat exchanger 6. The 1,2-propanediol containing phase P₂ and the hydrogen phase H₂ are then cooled further in the cooling region K_{2/2} (8). The composition which is cooled in this way is then transferred to a separator 9 in which the 1,2-propanediol containing phase P₂ is separated from the hydrogen phase H₂. The hydrogen in the hydrogen phase H₂ can then be returned to the reactor R₁ as a circulating gas after filling-up with hydrogen and an increase in pressure. A portion of the hydrogen gas is however introduced into the reactors R₁ and R₂ as quench gas via tubes (3) provided with holes.

The 1,2-propanediol containing phase P₂ which is separated off in the separator can immediately be used after stress relief, without further purifying, for preparing subsequent products, for example for preparing fatty acid esters.

### LIST OF REFERENCE NUMERALS

1 Supply for educt mixture of glycerol and hydrogen
2 First reactor R₁
3 Tube for the supply of quench gas
4 Cooling region K_{1/2}
5 Second reactor R₂
6 Heat exchanger
7 Heating device
8 Cooling region K_{2/2}
9 Separator

1. A process for preparing 1,2-propanediol by hydrogenation of glycerol by means of hydrogen gas, wherein glycerol is reacted with hydrogen in at least “i” fluidically interconnected reactors R₁ to Rᵢ each having a hydrogenation catalyst to form 1,2-propanediol, wherein the process comprises sequential steps from reactors Rᵢ through Rₙ wherein n as used herein is an integer in the range from 2 to i, and wherein the 1,2-propanediol containing phase Pₙ and hydrogen phase Hₙ are formed in the reactor Rₙ, said steps comprising:
introducing hydrogen gas and a glycerol phase P_{glycerol} into the first reactor R₁ and forming a first, 1,2-propanediol containing phase P₁ and a first hydrogen phase H₁ in the reactor R₁, and
introducing sequentially the 1,2-propanediol containing phase Pₙ₋₁ formed in the preceding reactor Rₙ₋₁ and hydrogen are introduced into each of the subsequent reactors Rₙ.
wherein the glycerol phase P_{glycerol} contains at least 60% by weight, based on the total weight of the glycerol phase P_{glycerol}, of glycerol.

2. The process according to claim 1, wherein each 1,2-propanediol containing phase Pₙ is cooled
i) within each reactor Rₙ, or
ii) in a region between the reactor Rₙ and Rₙ₊₁, or
iii) within each reactor Rₙ and in a region between the reactor Rₙ and the reactor Rₙ₊₁.

3. The process according to claim 2, wherein in the event of cooling of the 1,2-propanediol containing phase Pₙ between the reactor Rₙ and the reactor Rₙ₊₁, between these reactors a cooling region K_{n/n+1} is arranged, through which the 1,2-propanediol containing phase Pₙ issuing from the reactor Rₙ is cooled, with the hydrogen phase Hₙ, prior to entering the reactor Rₙ₊₁.

4. The process according to claim 3, wherein in the cooling region K_{n/n+1} the 1,2-propanediol containing phase Ppₙ issuing from the reactor Rₙ is cooled by at least about 10° C., with the hydrogen phase Hₙ, before they enter the reactor Rₙ₊₁.

5. The process according to claim 3, wherein in the cooling region K_{n/n+1} the 1,2-propanediol containing phase Pₙ issuing from the reactor Rₙ is cooled by at least about 25° C., preferably together with the hydrogen phase Hₙ, before they enter the reactor Rₙ₊₁.

6. The process according to claim 2, wherein in the event of cooling of the 1,2-propanediol containing phase Pₙ within each reactor Rₙ, the 1,2-propanediol containing phase Pₙ is cooled
I) by passing cold hydrogen gas into the reactor Rₙ, or
II) by a cooling device inside the reactor Rₙ, or
III) by passing cold hydrogen gas into the reactor Rₙ and by a cooling device inside the reactor Rₙ.

7. The process according to claim 1, wherein i is an integer from the range of from 2 to 10.

8. The process according to claim 1, wherein i is an integer from the range of from 2 to 5.

9. The process according to claim 1, wherein at least one of the reactors R₁ to Rᵢ has a catalyst charge.

10. The process according to claim 1, wherein at least one of the reactors R₁ to Rᵢ contains a heterogeneous, copper or chromium containing catalyst.

11. The process according to claim 10, wherein the heterogeneous, copper or chromium containing catalyst contains a heterogeneous copper chromite catalyst.

12. The process according to claim 1, wherein at least one of the reactors R₁ to Rᵢ is a tubular reactor.

13. The process according to claim 1, wherein the glycerol phase P_{glycerol} entering the reactor R₁ and the 1,2-propanediol phase Pₙ₋₁ entering the reactors R₂ to Rᵢ are present at least partly in liquid form.

14. The process according to claim 13, wherein in at least one of the reactors R₁ to Rᵢ the hydrogenation is carried out in such a way that the glycerol phase P_{glycerol} entering the reactor R₁ or the 1,2-propanediol containing phase Pₙ₋₁ entering the reactors R₂ to Rᵢ is passed in trickle bed operation in parallel or countercurrent flow with hydrogen over a catalyst charge.

15. The process according to claim 1, wherein in at least one of the reactors R₁ to Rᵢ the hydrogenation is carried out at a temperature in the range of from about 180 to about 220° C.

16. The process according to claim 1, wherein the glycerol is reacted in the reactor R₁ to about 20 to about 80 mol %.

17. The process according to claim 1, wherein the process additionally includes the process step of purifying the 1,2-propanediol containing phase Pᵢ so as to obtain pure 1,2-propanediol.

18. A 1,2-propanediol containing phase Pᵢ produced in accordance with the process according to claim 1.

19. A device for preparing 1,2-propanediol, containing at least “i” fluidically interconnected reactors R₁ to Rᵢ having a hydrogenation catalyst, wherein
a) between at least one pair of adjacent reactors Rₙ and Rₙ₊₁ wherein n as used herein is an integer from the range of from 1 to i, a cooling region K_{n/n+1} is provided, through which the 1,2-propanediol containing phase Pₙ issuing from the reactor Rₙ is cooled prior to entering the reactor Rₙ₊₁, wherein the 1,2-propanediol containing phase Pₙ is formed in the reactor Rₙ, or
b) within at least one reactor Rₙ a cooling device is provided, or
c) between at least one pair of adjacent reactors Rₙ and Rₙ₊₁, a cooling region K_{n/n+1} is provided, through which the 1,2-propanediol containing phase Pₙ issuing from the reactor Rₙ is cooled prior to entering the reactor Rₙ₊₁, and in at least one reactor Rₙ a cooling device is provided.

20. The device according to claim 19, wherein the device further comprises a fat cleaver which is fluidically connected to at least one of the reactors R₁ to Rᵢ.

21. The device according to claim 19, wherein at least one of the reactors R₁ to Rᵢ contains a heterogeneous, copper or chromium containing catalyst.

22. The device according to claim 21, wherein the heterogeneous, copper or chromium containing catalyst contains a heterogeneous copper chromite catalyst.

23. The device according to claim 19, wherein at least one of the reactors R₁ to Rᵢ is a tubular reactor.

24. The process according to claim 17, further comprising the use of a device containing at least “i” fluidically interconnected reactors R₁ to Rᵢ having a hydrogenation catalyst, wherein said device comprises
a between at least one pair of adjacent reactors Rₙ and Rₙ₊₁ wherein n as used herein is an integer from the range of from 1 to i, a cooling region K_{n/n+1} is provided, through which the 1,2-propanediol containing phase Pₙ issuing from the reactor Rₙ is cooled prior to entering the reactor Rₙ₊₁, wherein the 1,2-propanediol containing phase Pₙ and hydrogen phase Hₙ are formed in the reactor Rₙ,
b) within at least one reactor Rₙ a cooling device is provided, or between at least one pair of adjacent reactors Rₙ and Rₙ₊₁, a cooling region K_{n/n+1} is provided, through which the containing phase Pₙ issuing from the reactor Rₙ is cooled prior to entering the reactor Rₙ₊₁ and in at least one reactor Rₙ a cooling device is provided.

25. A process for preparing a compound comprising a group having at least one ether group, or at least one ester group, or at least one amino group, or at least one urethane group, or at least two thereof, including the process steps:
ia) providing a 1,2-propanediol containing phase Pᵢ using a process comprising
introducing hydrogen gas and a glycerol phase P_{glycerol} into the first reactor R₁ and a first 1,2-propanediol containing phase P₁ and a first hydrogen phase H₁ are formed in the reactor R₁, and
introducing sequentially the 1,2-propanediol containing phase Pₙ₋₁ formed in the preceding reactor Rₙ₋₁ and hydrogen into each of the subsequent reactors R and 1,2-propanediol containing phase Pₙ and hydrogen phase Hₙ are formed in the reactor Rₙ, or
ib) providing a purified 1,2-propanediol produced according to the process comprising
introducing hydrogen gas and a glycerol phase P_{glycerol} into the first reactor R₁ and a first 1,2-propanediol containing phase P₁ and a first hydrogen phase H₁ are formed in the reactor R₁.
introducing sequentially the 1,2-propanediol containing phase Pₙ₋₁ formed in the preceding reactor Rₙ₋₁ and hydrogen into each of the subsequent reactors Rₙ and 1,2-propanediol containing phase Pₙ and hydrogen phase Hₙ are formed in the reactor Rₙ, and
purifying the 1,2-propanediol containing phase Pᵢ so as to obtain pure 1,2-propanediol; and
ii) reacting the 1,2-propanediol contained in the 1,2-propanediol containing phase P₁ or the purified 1,2-propanediol with a compound comprising a group having at least one active hydrogen atom, or at least one epoxide group, or at least one ester group, or at least one isocyanate group.

26. The process according to claim 25, wherein in process step ii) the 1,2-propanediol contained in the 1,2-propanediol containing phase P₁ or the purified 1,2-propanediol is reacted with a compound having at least one carboxylic acid group so as to obtain a compound having at least one ester group.
